(19) United States
(12) Patent Application Publication
(10) Pub. No.: US 2012/0041302 A1
(43) Pub. Date: Feb. 16, 2012

(54) PORTABLE IMAGING SUBJECT CARTRIDGE

(75) Inventors: David G. NILSON, Walnut Creek, CA (US); Bradley W. RICE, Danville, CA (US); Heng XU, Union City, CA (US); Tamara L. TROY, San Francisco, CA (US)

(73) Assignee: CALIPER LIFE SCIENCES, INC., Alameda, CA (US)

(21) Appl. No.: 12/857,469

(22) Filed: Aug. 16, 2010

(51) Int. Cl. A61B 5/05

(52) U.S. Cl. 600/427

(57) ABSTRACT

An imaging subject handling system includes multiple portable imaging subject cartridges that optionally compress and anesthetize living imaging subjects therein during imaging, multiple receiving bases installed within separate imaging systems that interface with the cartridges, and multiple gas delivery systems that deliver an anesthetic gas flow to an installed cartridge. The cartridges include a gas delivery interface that accepts the anesthetic gas flow and provides it to the imaging subject, and one or more walls, a bottom and a top defining a closed interior that retains the anesthetic gas therewithin while the cartridge is in transport between separate imaging systems. The walls, bottom and/or top can be optically transparent and radiolucent to facilitate imaging. The cartridge can include a locking mechanism, a nose cone for the imaging subject, and co-registration features located on outside surfaces to facilitate the merging of images within software from multiple separate imaging systems.

### TECHNICAL FIELD

The present invention relates generally to systems and methods for obtaining multiple types of internal images of a subject, and more particularly to the transfer of an imaging subject between different imaging systems.

### BACKGROUND

There are currently numerous non-invasive imaging techniques that can be used to produce images of an imaging subject. Such techniques include, for example, X-rays, magnetic resonance imaging (“MRI”), computed tomography (“CT”) scans, ultrasound, and the like. In addition, various non-invasive optical imaging techniques such as bioluminescence and fluorescence can be used to produce optical images of animal subjects, such as in the areas of medical research, pathology, drug discovery and development, and the like. Each such imaging technique has advantages and disadvantages that make it useful for different imaging applications. Some techniques are well suited to provide spatial or anatomical information for internal parts, while others are more suited to provide functional information for an activity of interest within a subject being imaged. Due to the differing advantages of different types of imaging systems, it has become increasingly desirable to combine the differing outputs and strengths of multiple imaging systems for a single imaging subject.

Unfortunately, the cost of combining various traditional imaging systems into a single system is prohibitive, even if numerous practical complications that inhibit such a combination imaging system environment could be overcome. As a result, a single imaging subject is often transferred between different imaging systems in order to obtain various types of imaging results that can then be utilized to better effect in combination with each other. In ideal circumstances, the transfer of an imaging subject from one imaging system to another would result in the imaging subject being in the exact same position for imaging in both imaging systems. In such circumstances, the various images from the different imaging systems can then be overlaid, superimposed or otherwise manipulated in a manner that results in the ability to view or interpret the images together with a fair degree of reliability.

As might be expected, however, the transfer of an imaging subject can be a less than ideal venture, as it is often difficult to maintain the spatial accuracy provided by each system without compromise. Imaging subject transfer issues can include, for example, jostling or bumping by the person or apparatus moving the subject between imaging systems. Further problems can arise where the imaging subject is a living animal or specimen, such as a mouse, that would ordinarily be inclined to move on its own during the transfer. Substantial changes in imaging subject positioning, muscle flexing and the like during a transfer between imaging systems can then result in images from the second and/or subsequent imaging systems that do not overlap well with images from the first and/or prior imaging systems.

While many systems and methods for providing multiple types of internal images of a subject have generally worked well in the past, there is always a desire to provide new and improved ways to obtain such internal images. In particular, what are desired are more reliable systems and methods for transferring imaging subjects between different imaging systems without substantially disturbing or altering the positions of the imaging subjects.

### SUMMARY

It is an advantage of the present invention to provide systems and methods that facilitate the ready transfer of living imaging subjects between different imaging systems without substantially disturbing or altering the positions of the imaging subjects. Such living imaging subject transfers between systems can be accomplished at least in part through the use of a portable imaging subject cartridge that holds the imaging subject in a stable and optionally compressed state while also providing an anesthetic gas around the anesthetized imaging subject.

In various embodiments, a portable imaging subject cartridge can be adapted to contain and anesthetize a living imaging subject therein during imaging processes within and transport between separate imaging systems. The portable imaging subject cartridge can include a gas delivery interface including a gas inlet and gas exhaust, as well as one or more walls, a bottom and a top that together define a closed interior and operate to retain substantially all of an anesthetic gas therewithin while the portable imaging subject cartridge is in transport between separate imaging systems. The gas delivery interface can be adapted to accept an anesthetic gas flow from an outside source and provide the anesthetic gas to the living imaging subject while the portable imaging subject cartridge is within one of the separate imaging systems. In addition, at least one of the walls, bottom and top can include an optically transparent and radiolucent region therethrough, in order to facilitate imaging of the imaging subject while it is within the cartridge. In various embodiments, the living imaging subject can be a mouse, although other imaging subjects are also possible.

In various detailed embodiments, the portable imaging subject cartridge can also include one or more co-registration features located on the outer surface of at least one of the walls, bottom and/or top. Such a co-registration feature or features can be adapted to facilitate the accurate and repeated positioning of the portable imaging subject cartridge inside multiple separate imaging systems, such that the obtained images from the different imaging systems are more easily overlaid with respect to each other during image processing.

Additional features can include the top comprising a separable lid that can be removable from the remainder of the portable imaging subject cartridge, an internal nose cone adapted to receive the nose of the living imaging subject and deliver the anesthesia gas from the gas delivery interface thereto, and both of the bottom and top comprising substantial transparent regions, whereby visible to near infrared light from a light source located beneath the cartridge shines through the cartridge and is receivable at an imaging device located above the cartridge. In addition, the cartridge walls can be dimensioned such that the living imaging subject is compressed between the bottom and top when the cartridge is closed and the living imaging subject is contained therein. In various embodiments, the cartridge can include no ferrous metal parts, such that imaging can be conducted in MRI systems. Also, the portable cartridge can be adapted to be carried manually during transport between separate imaging systems.

In various further embodiments, an imaging subject handling system can include a plurality of portable imaging subject cartridges, such as those provided in detail above. In addition, the imaging subject handling system can also include a receiving base adapted to be installed within a chamber of a separate imaging system, wherein the receiving base is configured to receive and interface with the various portable imaging subject cartridges. The handling system can also include a gas delivery system coupled to the receiving base and configured to deliver the anesthesia gas flow to and receive exhaust from the gas delivery interface of a portable imaging subject cartridge installed at the receiving base. Further receiving bases can also be adapted for installation in various additional imaging systems, such that the plurality of portable cartridges can be transported between and installed within the bases in the different imaging systems. In addition, the receiving base can include a gas processing component adapted to receive the anesthetic gas flow from the outside source and deliver the anesthetic gas flow to the gas delivery interface of an installed portable cartridge. The receiving base can also comprise a substantially flat surface having an opening therethrough adapted to receive a portable cartridge therein.

In still further embodiments, various methods of imaging a living imaging subject in multiple different imaging systems, can include the process steps of placing the living imaging subject within a portable imaging subject cartridge such as that provided above, compressing the living imaging subject between the top and bottom of the portable imaging subject cartridge, and closing off the interior of the portable imaging subject cartridge such that ambient air outside the portable imaging subject cartridge does not substantially mix with gas inside the interior or the portable imaging subject cartridge. For each imaging system where imaging of the living imaging subject is desired, further process steps can then include placing the portable imaging subject cartridge within the imaging system, providing a flow of anesthetic gas to the interior of the portable imaging subject cartridge while the portable imaging subject cartridge is therewithin, conducting imaging on the living imaging subject while the living imaging subject is compressed within the portable imaging subject cartridge and the portable imaging subject cartridge is within the imaging system, and removing the portable imaging subject cartridge from the imaging system.

Various methods can also include the step of transporting the portable imaging subject cartridge from one imaging system to the next imaging system, wherein a portion of the anesthetic gas remains within the interior of the portable imaging subject cartridge during the step of transporting. Further process steps that can be conducted while a cartridge is being installed into an imaging system can include registering the exact location of the portable imaging subject cartridge by reading one or more co-registration features located on the outer surface of at least one of the one or more walls, bottom and top of the portable subject imaging cartridge.

Other apparatuses, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The included drawings are for illustrative purposes and serve only to provide examples of possible structures and arrangements for the disclosed inventive systems and methods for transferring imaging subjects between different imaging systems. These drawings in no way limit any changes in form and detail that may be made to the invention by one skilled in the art without departing from the spirit and scope of the invention.

FIG. 1A illustrates in block diagram format an exemplary system adapted to provide handling and multiple different modes of imaging for an internal portion of an imaging subject.

FIG. 1B illustrates in front perspective view an exemplary imaging system adapted to produce a 2-D or 3-D representation of a light source located inside an imaging subject.

FIG. 1C illustrates in front perspective view an exemplary MRI system.

FIG. 1D illustrates in overview perspective view an exemplary system adapted to provide imaging subject handling as well as multiple different modes of imaging using the imaging systems of FIGS. 1B and 1C.

FIG. 2 illustrates in top perspective view an exemplary portable imaging subject cartridge with its lid open and a superimposed mouse imaging subject disposed therein according to one embodiment of the present invention.

FIG. 3 illustrates in top perspective view the exemplary portable imaging subject cartridge of FIG. 2 and a receiving base therefor according to one embodiment of the present invention.

FIG. 4 illustrates in obverse perspective view the exemplary portable imaging subject cartridge of FIG. 2 with its lid closed as it is being docked into the receiving base of FIG. 3 according to one embodiment of the present invention.

FIG. 5 illustrates in top perspective view an exemplary portable imaging subject cartridge docked into a receiving base and having a dummy imaging subject disposed therein according to one embodiment of the present invention.

FIG. 6 illustrates in top perspective view an exemplary portable imaging subject cartridge docked into a receiving base having an alternative gas porting arrangement according to one embodiment of the present invention.

FIG. 7 illustrates in top perspective view an exemplary portable imaging subject cartridge and two different types of receiving bases having different co-registration features therefor according to one embodiment of the present invention.

FIG. 8A illustrates in top perspective view an exemplary portion of a portable imaging subject cartridge handling system according to one embodiment of the present invention.

FIG. 8B illustrates in top plan view three exemplary fiducial co-registration patterns for a multi-modal imaging system according to various embodiments of the present invention.

FIG. 9A illustrates in side cross-sectional view an imaging subject within an exemplary portable imaging subject cartridge placed atop a triangular fiducial co-registration pattern according to one embodiment of the present invention.

FIG. 9B illustrates an exemplary CT image background result from the triangular fiducial co-registration pattern of FIG. 9A according to one embodiment of the present invention.

FIG. 9C illustrates an exemplary set of x-points corresponding to the identification of the edge of the feature of FIG. 9B using image processing software.

FIG. 10 presents a flowchart of an exemplary method of transferring an imaging subject between different imaging systems according to one embodiment of the present invention.

### DETAILED DESCRIPTION

Exemplary applications of apparatuses and methods according to the present invention are described in this section. These examples are being provided solely to add context and aid in the understanding of the invention. It will thus be apparent to one skilled in the art that the present invention may be practiced without some or all of these specific details. In other instances, well known process steps have not been described in detail in order to avoid unnecessarily obscuring the present invention. Other applications are possible, such that the following examples should not be taken as limiting.

In the following detailed description, references are made to the accompanying drawings, which form a part of the description and in which are shown, by way of illustration, specific embodiments of the present invention. Although these embodiments are described in sufficient detail to enable one skilled in the art to practice the invention, it is understood that these examples are not limiting; such that other embodiments may be used, and changes may be made without departing from the spirit and scope of the invention.

The invention relates in various embodiments to the reliable transfer of an imaging subject between different imaging systems, with the transfer being conducted in a manner such that the imaging subject is disturbed as little as possible. Such an imaging subject can be an animal, such as a mouse or other lab animal, and can be alive during the imaging and transfer processes. In particular, the present invention relates to a portable imaging subject cartridge or carrier. In a detailed example, such as that which is provided herein for purposes of illustration, such a portable cartridge can be a portable “Mouse Imaging Shuttle” that is adapted to lightly press flatten and anesthetize the mouse between top and bottom transparent plates that operate to form a full enclosure for the mouse. The anesthetic keeps the mouse sedated during transfer and imaging processes, while the press flattening operates both to limit the amount of movement or jostling during transfer and also to provide better images. Of course, other imaging subjects can also be similarly used in a system that is the same or substantially similar to that which is disclosed herein, and it is contemplated that any and all such suitable alternative imaging subjects can also be used.

### Introduction

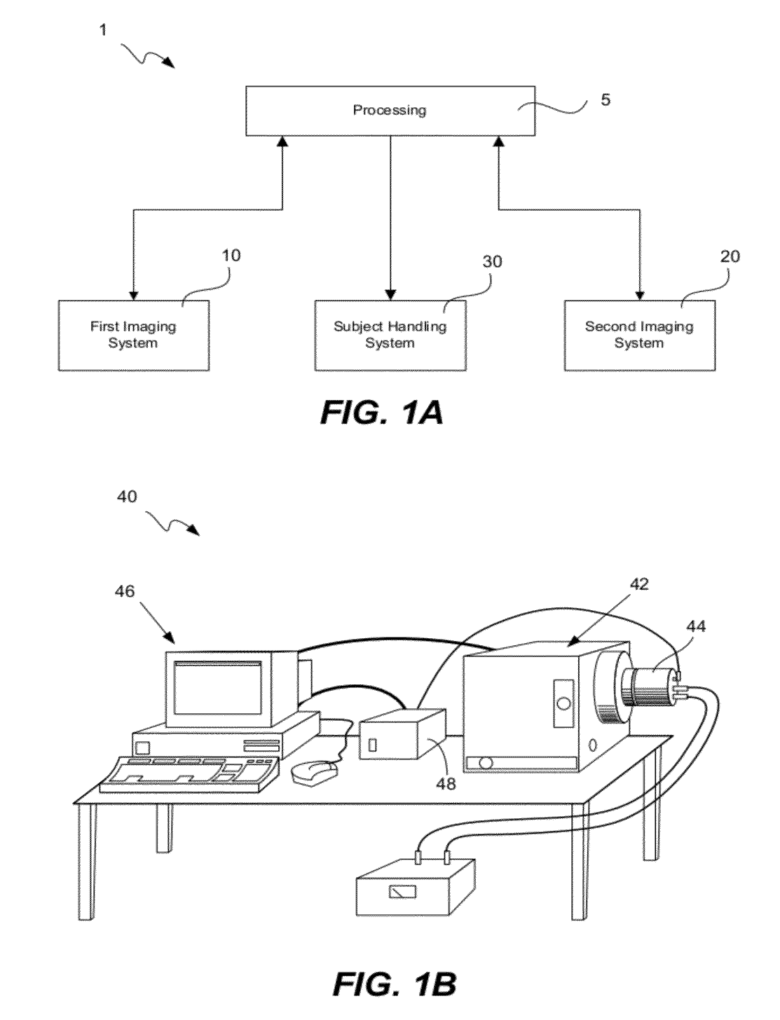

Referring first to FIG. 1A, an exemplary system adapted to provide handling and multiple different modes of imaging for an internal portion of an imaging subject is shown in block diagram format. Multi-modal imaging system 1 comprises a first imaging system 10, a second imaging system 20, a subject handling system 30, and at least one processor 5 adapted to control one or more of the imaging system(s), subject handling system, and/or to process together multiple images obtained from the different imaging systems. The subject handling system 30 transfers a subject being imaged between the first imaging system 10 and the second imaging system 20. While the present invention is primarily described herein with respect to combining data from two imaging systems, multi-modal imaging system 1 may include more than two imaging systems, and the illustrative embodiment is not meant to limit the number of imaging systems that are so combined. Details of a multi-modal imaging system are described in commonly owned U.S. Pat. No. 7,190,991 entitled “Multi-Mode Internal Imaging,” which is incorporated herein in its entirety and for all purposes.

First imaging system 10 and second imaging system 20 can both employ any one of a variety of imaging modes, and each imaging system 10, 20 preferably uses a mode of imaging that is different that the other imaging system(s) in multi-modal imaging system 1. Exemplary imaging systems include, for example, various light imaging systems such as photographic, bioluminescent, and/or structured light imaging systems, as well as other types of imaging systems, such as MRI systems; CT systems; CAT scan systems; X-ray systems; ultrasound systems; nuclear medicine imaging systems such as positron emission tomography (“PET”) systems, single photon emission computed tomography (“SPECT”) systems, cardiovascular imaging systems, and bone scanning systems, among other possible imaging systems.

First imaging system 10 and second imaging system 20 may produce spatial and/or functional information. Spatial information refers to information that contributes to a 2-D (pictorial) or 3-D geometric description of the subject or its internal portions. A spatial representation provides a user with a 2-D or 3-D pictorial reference of the specimen. A 3-D geometric description typically comprises a reconstruction manufactured by processing data from multiple 2-D images. Functional information refers to information that contributes an item or activity of interest within the subject. In one embodiment, one of the included imaging systems produces a 2-D or 3-D representation of a bioluminescent light source inside a mouse. The bioluminescent source may correspond to a wide variety of physiological issues being tracked or tested within the mouse, such as progress tracking of a particular cancer within a mouse. Some imaging applications include analysis of one or more representations of light emissions from internal portions of a specimen superimposed on a spatial representation of the specimen. The luminescence representation indicates portions of a specimen where an activity of interest may be taking place.

FIG. 1B illustrates in front perspective view an exemplary imaging system adapted to produce a 2-D or 3-D representation of a light source located inside an imaging subject. Light imaging system 40, which can correspond to first imaging system 10 from FIG. 1A, may be used for a variety of imaging tasks, including the capture of photographic, luminescent and structured light images. Light imaging system 40 can be a light imaging system that involves the capture of low intensity light—often on the order of about 10³ to about 10¹⁰ photons/second/centimeter squared/steradian—from a light-emitting subject. The low intensity light may be emitted from any of a variety of light sources within the imaged subject. For example, the light source may correspond to luciferase expressing cells within a living specimen, such as a mouse. The light source indicates portions of the sample, such as traced molecules in a particular portion of a living specimen, where an activity of interest may be taking place. As will be readily appreciated, such light imaging can involve bioluminescent imaging, fluorescent imaging, and/or any other suitable type of imaging.

As shown, light imaging system 40 can include an imaging chamber 42 adapted to receive a light-emitting sample in which low intensity light is to be detected. A high sensitivity camera 44, such as an intensified or a charge-coupled device (“CCD”) camera, can be coupled with the imaging chamber 42. Camera 44 can be capable of capturing luminescent, photographic (i.e., reflection based images) and structured light images of an imaging subject within imaging chamber 42. A computer 46 and its inclusive processor 5 working with light imaging system 40 may perform processing and imaging tasks such as obtaining, analyzing and manipulating 2-D or 3-D light source representations. An image processing unit 48 optionally interfaces between camera 44 and computer 46, and can be used to help generate composite images, such as combination photographic and luminescent images.

Light imaging systems 40 suitable for use with the present invention are available from Caliper Life Sciences of Hopkinton, Mass. Several light imaging systems suitable for use with the present invention are described in commonly owned U.S. Pat. No. 7,113,217 entitled “Multi-View Imaging Apparatus,” which is incorporated by reference herein for all purposes. 3-D imaging systems suitable for use with the present invention are further described in commonly owned U.S. Pat. No. 7,616,985 entitled “Method and Apparatus for 3-D Imaging of Internal Light Source,” which is also incorporated by reference herein for all purposes. Various approaches to generating composite photographic/luminescence images, such as might be desired from the foregoing systems are described in U.S. Pat. No. 5,650,135 issued to Contag et al. on Jul. 22, 1997, which is incorporated herein in its entirety and for all purposes.

FIG. 1C illustrates in front perspective view an exemplary MRI system. MRI system 50, which can correspond to second imaging system 20 from FIG. 1A, typically comprises an MRI machine 52 and at least one processor (not shown) in digital communication with the electronic components of the MRI system. MRI machine 52 may employ a suitable magnet 54 from about 0.5-tesla to about 60.0-tesla, or about 5,000 to about 600,000 gauss. Using radio wave pulses of energy the MRI machine 52 scans the subject and portions inside to build up a 2-D or 3-D map of tissue types. As shown, MRI machine 52 can include a horizontal tubular cavity 54 that passes through a magnet 56, as is generally well known. A subject 60 to be imaged rests on a portable stage 72 that is moved into tubular cavity 54. Imaging subject handling system 70 can be adapted to positions stage 72 and subject 60 in tubular cavity 54. A processor receives the magnetic output and uses this to create 2-D images or 3-D models. A processor may also combine 2-D or 3-D spatial information provided by MRI system 50 with 3-D functional information provided by the light imaging system 40.

Although an MRI system 50 has been shown and discussed for purposes of illustration, it will be readily appreciated that numerous other types of imaging systems could also be used along with or in lieu of an MRI system. For example, a CT system could be used instead of an MRI system, and might even be preferable in various circumstances. In such instances, the basic nature of a CT system, and/or any other suitable additional or replacement type of imaging system, will be readily understood by those skilled in the art, such that a brief description of each basic type of imaging system need not be set forth herein.

FIG. 1D illustrates in overview perspective view an exemplary system adapted to provide imaging subject handling as well as multiple different modes of imaging using the imaging systems of FIGS. 1B and 1C. Imaging subject handling system 70 can be configured to move a portable stage 72 between and within the interior cavities of both light imaging system 40 and MRI system 50. As shown in FIG. 1D, imaging subject handling system 70 can include a portable stage 72 having an imaging subject 60. Portable stage 72 is adapted to be held by manipulator 74, which in turn moves along track 76. Track 76 can be adapted to transport imaging subject 60 and portable stage 72 to and between different imaging systems 40, 50, and allows large movement for the subject handling system 70 over large distances, such as between rooms. Again, further details of a multi-modal imaging system and an imaging subject handling system therefor are described in commonly owned U.S. Pat. No. 7,190,991 entitled “Multi-Mode Internal Imaging,” which is incorporated herein in its entirety and for all purposes.

As will be readily appreciated, while the use of an automated imaging subject handling system such as system 70 may work well to reduce imaging subject disturbances between imaging systems, such a handling system might be considered bulky, cumbersome, costly and/or slow depending upon various applications. In addition, the use of only the disclosed simple portable stage 72 to transport a living imaging subject, such as a mouse, between imaging systems does little to restrict imaging subject movement or otherwise account for the ability of the living imaging subject to wake up from the effects of anesthesia. Of course, waking up from the effects of anesthesia would typically be detrimental, as the imaging subject would then be inclined to move around or otherwise change or disturb its position that was used during imaging in one or more prior imaging systems. As such, additional and/or alternative imaging subject handling systems adapted to transport an imaging subject between imaging systems while minimizing imaging subject disturbances could be useful.

### Mouse Imaging Shuttle

It is specifically contemplated that the additional imaging subject handling devices and systems disclosed below can be used in conjunction with and/or in lieu of the automated imaging subject handling system 70 described above. In particular, a primary feature of an alternative living imaging subject handling system is a portable imaging subject cartridge, which can be placed on and handled by a portable stage such as stage 72 and also transported by a manipulator 74 along a track 76, such as that which is set forth above. Alternatively, the portable imaging subject cartridge and other various system components described below can be used without one or more of the components of system 70 above. For example, the portable imaging subject cartridge provided herein can be carried by hand and otherwise handled manually while still preserving the position of the imaging subject contained therein.

As a particular example, a Mouse Imaging Shuttle (“MIS”) can be one form of a portable imaging subject cartridge. Although the detailed description provided herein is made with respect to the specific example of a MIS and its related supporting components, it will be readily appreciated that other forms of portable imaging subject cartridges and supporting components can include similar features and advantages, such as for use with imaging subjects other than mice. In particular, such other portable imaging subject cartridges can, similar to the MIS described in detail herein, hold an imaging subject in a stable compressed state therein while also providing an anesthetic gas around the anesthetized imaging subject.

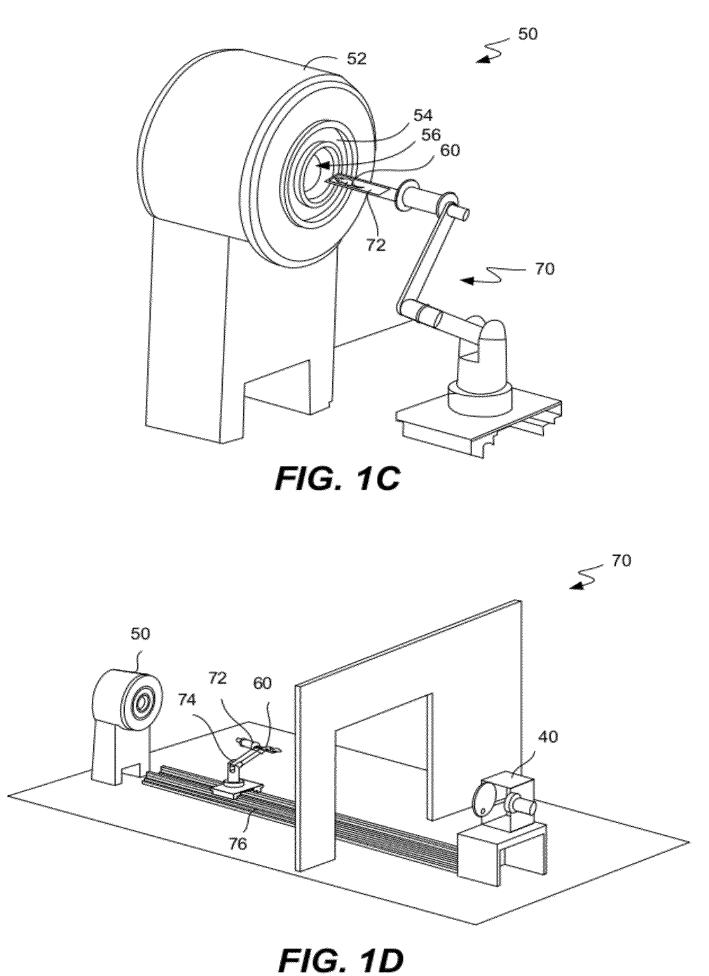

Turning next to FIG. 2, an exemplary portable imaging subject cartridge with its lid open and a superimposed mouse imaging subject disposed therein is illustrated in top perspective view according to one embodiment of the present invention. Portable imaging subject cartridge 100 can be adapted to contain a living imaging subject 61 therein. Such a living imaging subject can be a mammal, for example, and in a particular embodiment can be a mouse 61. The mouse 61 is preferably anesthetized while it is contained within the cartridge 100, or at least from the time of the first imaging process through the final imaging process. As shown, portable imaging subject cartridge 100 can include a body portion 110, a separable top or lid 120, a locking mechanism 130 adapted to fasten the lid to the body portion and a gas delivery interface 140 adapted to accept an anesthetic gas flow from an outside source and provide the gas to the enclosed portion of the cartridge.

Although the portable imaging subject cartridge is illustrated as being partially open, it will be readily appreciated that the lid 120 can be removable from the remainder of the cartridge and can also be locked into place when it is placed down against the walls 112 of body portion 110. Locking mechanism 130 can comprise a swiveling or pivoting component having a shaft 132 that is adapted to pivot up into a slot 124 in the lid 120 and an enlarged head 132 that locks the lid in place. In some embodiments, the head 134 and shaft 132 can include a threaded relationship, such that the locking mechanism 130 can be tightened against the lid 120 to squeeze the lid more tightly against the body portion 110. Body portion 110 can also include a raised lip 116 adapted to receive the edge of the lid 120 at the end opposite the locking mechanism 130. Raised lip 116 can be adapted to restrict both lateral and upward movement of the lid 120 when the lid is installed against the body portion 110. Similarly, shaft 132 restricts lateral movement of the lid in the other direction, while the underside of head 134 restricts upward motion of the lid when the locking mechanism 130 is engaged. An optional gasket (not shown) may also be used between the interfacing parts of the body and lid to effect a better seal if desired.

Removable lid 120 can include a substantially transparent region 122, such that the imaging subject 61 contained therein can be readily imaged by a camera or other imaging device located above the portable cartridge 100. In addition, the body portion 110 can similarly include a substantially transparent region 114 on the floor and/or side wall or walls thereof, such that one or more light sources can shine light through the portable cartridge 100 to facilitate imaging. The transparent bottom region 114 can also facilitate improved high density scans and little to no edge effects for CT scans.

With respect to the term “transparent,” it will be understood that this term generally refers to that which is needed by the various types of imaging systems that may be used in an overall multi-modal system. Thus, the term means optically transparent with respect to optical imaging system, and can also mean radiolucent (i.e., transparent to x-rays) where CT systems (i.e., x-ray transparency) are used in a given overall system, for example. For purposes of optical transparency, the transparent regions 114, 122 can be comprised of glass, plastic or any other material suitable for transmitting light therethrough. In some embodiments, the entire portable cartridge can be made out of glass, plastic, or another suitable transparent material. Where CT systems (i.e., x-rays) are involved, however, then glass may not be the best material for such transparent regions. Accordingly, a suitable plastic or other optically transparent and radiolucent material can be used for multi-modal systems that involve CT imaging systems and optical imaging systems. In addition, the entire portable cartridge 100 is preferably comprised of no ferrous metal parts, such that the cartridge can be used in MRI systems.

Gas delivery interface 140 can include a gas inlet and gas exhaust for porting to outside gas lines. As such, gas delivery interface 140 can be adapted to accept an anesthetic gas flow from an outside source and provide the anesthetic gas to the living imaging subject 61 to keep the subject sedated and/or anesthetized during imaging and transport between systems. In various embodiments, the overall multi-modal imaging system can include multiple disparate imaging systems, some or all of which are adapted to serve as the outside source that provides an anesthetic gas to the imaging subject 61 inside the portable cartridge 100 while the portable cartridge is contained within the imaging system.

To facilitate gas delivery to the imaging subject 61, a nose cone 142 can be located inside the closed interior defined by the walls, bottom and lid of the cartridge 100. As will be readily appreciated, the living imaging subject can be positioned within the cartridge such that its nose is located within nose cone 142. Anesthetic gas can preferably flow into the enclosed interior region by way of the nose cone, circulate within the interior region, and then be exhausted out one or more exhaust ports, which can be located inside the cartridge and to the side of the nose cone 142, for example. In addition, one or more small relief holes 144 can be present at various locations on the walls, lid and/or floor of portable cartridge 100. Such a relief hole or holes 144 can facilitate a suitable pressurization of the cartridge with respect to the ambient air on the outside of the portable cartridge, and can also facilitate an appropriate flow of anesthetic gas throughout the internal cavity of the portable cartridge. For example, relief holes 144 can facilitate a negative pressure volume, such that a suffocating vacuum or near vacuum is not produced when the anesthetic gas is pumped out of the portable cartridge. Although sealing the portable cartridge from ambient is not thought to be necessary, it may be desirable to design relief holes 144 and the gas delivery systems so as to allow for some mixing with ambient while in an imaging system, while minimizing mixing with ambient while the portable cartridge is in transit.

One notable feature for portable imaging subject cartridge 100 is that the depth of the cartridge (i.e., height of the sidewall(s) 112) can be dimensioned such that the living imaging subject 61 can optionally be squeezed or compressed between the lid 120 and bottom portion 114 when the lid is installed and the cartridge is closed, if desired. One advantage for doing so is that compression of the imaging subject 61 can improve the ability to image one or more items that are internal to the subject, since compressing the animal tissue tends to bring such internal components closer to a surface region of the imaging subject. Compression of the imaging subject can also enhance the signal to noise ration for many imaging systems, and also produces a more uniform surface region to evaluate the imaging results. Another notable advantage to compressing the imaging subject is that such compression tends to restrict subject movement better than an unrestricted imaging subject, which can be helpful as the cartridge and imaging subject are transported between different imaging systems.

In various embodiments, different portable imaging subject cartridges within an overall system can have varying depths (i.e., heights), so as to account for different sizes in imaging subjects. For example, some portable cartridges may have a depth of about one-half inch for smaller mice, while other portable cartridges can have a depth of about one inch for larger mice. Of course, other smaller, larger and in between depth dimensions may also be used, as may be desired. Such varying degrees of depth can allow for slight or more substantial compression of the imaging subject during imaging, as may be desired. Alternatively, not squeezing the imaging subject can also be an option, in which case selecting an imaging subject cartridge having a larger depth for a given imaging subject would be a possibility. Preferably, the various receiving or docking components within the various imaging systems are adapted to receive each of the various imaging subject cartridges regardless of the varying cartridge depths.

Another noteworthy feature on the disclosed portable imaging subject cartridges can involve the use of one or more orientation features 150 located on one or more of the outside surfaces of the portable cartridge 100. As shown, a front corner of portable cartridge 100 includes a triangular shaped orientation shape 150. These orientation features, along with the overall shape and dimension of the cartridge itself can be used to facilitate the accurate repeated positioning of the portable imaging subject cartridge 100 from one imaging system to another. That is, where an imaging system includes suitable mating equipment adapted to mate with the various orientation shapes or features on the outer surfaces of portable cartridges installed therein, such features can then be used to calibrate the positioning of a given cartridge and the imaging subject contained therein when data is combined from different imaging systems for the same imaging subject in a given imaging session. Alternative forms of co-registration marks or shapes can also be used in a given system, and differing types can be used in the same overall multi-modal imaging system for a single cartridge, as set forth in greater detail below.

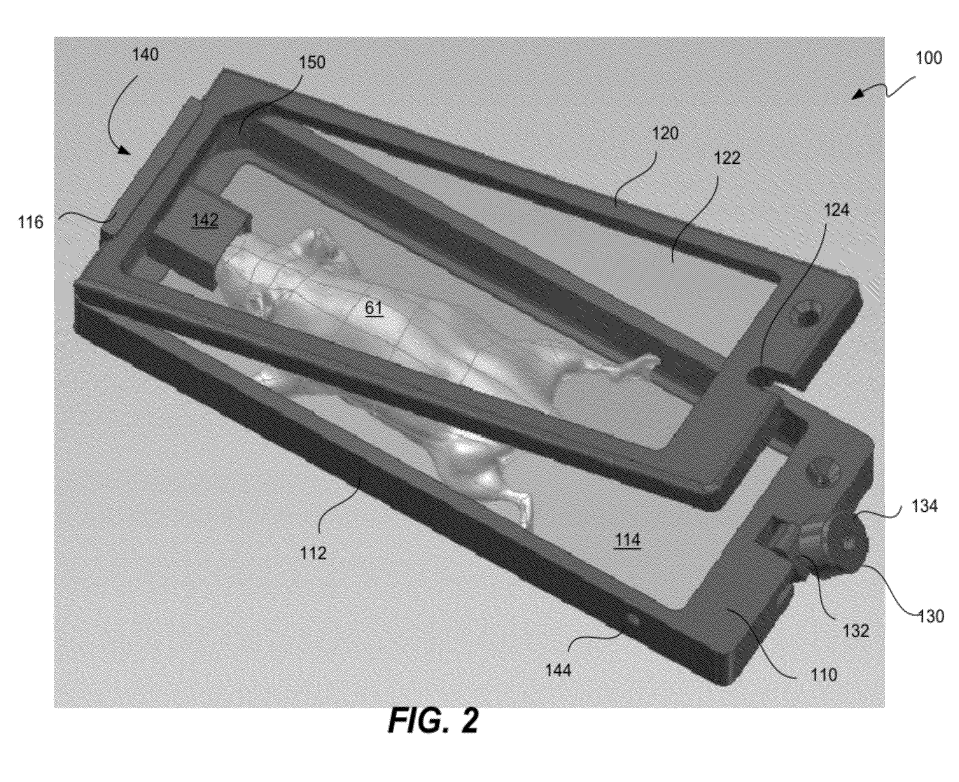

As will be readily appreciated, further components can also be useful in facilitating the streamlined use of a system of portable imaging subject cartridges, such as the one described above. Continuing now with FIG. 3, the exemplary portable imaging subject cartridge of FIG. 2 and a receiving base therefor are shown in top perspective view according to one embodiment of the present invention. Imaging subject handling system 200 includes portable imaging subject cartridge 100 and a receiving base 210 adapted for use therewith. While the portable cartridge 100 contains the imaging subject and is adapted to be transported between imaging systems, the receiving base 210 can generally be installed or stationed within a given imaging system and adapted to interface with various portable cartridges. Such installation of the receiving base 210 within an imaging system can be permanent or semi-permanent, and can be effected by way of glue, adhesive, fasteners, clips and/or any other suitable retaining mechanisms. The receiving base can be installed on a floor or a movable stage of an imaging system, among other possible locations.

As shown, receiving base 210 can include a substantially flat surface 212 having an opening 214 therethrough that is dimensioned to receive portable cartridge 100 therein. Receiving base 210 can also include or at least be adapted to interface with a gas processing component 220 adapted to receive the anesthetic gas flow and deliver the gas flow forward to the gas delivery interface 140 of the cartridge 100. A gasket 222 or other interfacing component can help to facilitate the porting of the gas processing component 220 to the gas delivery interface 140 when the portable cartridge 100 is installed. As shown, gasket 222 includes three holes therein, so as to provide gas inlet, gas exhaust, and also guide the alignment of interfacing items. A gas inlet hose 224 and gas exhaust hose 226 can also be coupled to gas processing component 220, as will be readily appreciated.

It will be understood that while anesthetic gas can be readily provided to, circulated within and exhausted from a portable cartridge 100 while the cartridge is installed in a receiving base 210, such gas provisions can typically cease once the portable cartridge is removed from the receiving base and imaging system. Although no fresh anesthetic gas is being provided to the interior region of the portable cartridge once the cartridge is removed from an imaging system, a sufficient amount of residual anesthetic gas remains within the cartridge to permit the living imaging subject to remain anesthetized or sedated for a short time period. Such a short time period can be on the order of one to five minutes or more, and can be sufficient to enable the transporting of the cartridge to and installment within a different imaging system having another receiving base and anesthetic gas supply installed therein, whereupon anesthetic gas delivery can then be resumed at the next system.

In addition, it can be seen that portable cartridge 100 can have a triangular orientation feature or shape 150 in one corner of the portable cartridge, and that receiving base 210 can have a matching or mating orientation feature or shape 250 in one corner of opening 214. Such mating orientation shapes 150, 250 can be arranged such that there is only one possible arrangement of the portable cartridge 100 within opening 214. Further, the orientation shapes 150, 250 can be designed or arranged such that a precise location of the portable cartridge within the respective imaging system can be achieved. For example, where triangular shaped mating features are used, such as orientation features 150, 250, then the shapes of the mating components requires the portable cartridge 100 to be in an exact location when the cartridge is placed flush against feature 250, as will be readily appreciated.

Imaging of the imaging subject within portable cartridge 100 between multiple imaging systems can then be consistent based on such a precise locating of the cartridge. For example, the overall opening 214 of receiving base 210 can have its location within an optical imaging system calibrated with the exact location of a respective CCD camera. Where the portable cartridges 100 to be used with base 210 are precisely dimensioned such that each cartridge fits snugly within opening 214, then the exact position of the cartridge is known within the optical imaging system. This is particularly true where there one or more sets of mating orientation features 150, 250 that prevent any backwards, upside-down or other incorrect positioning of a cartridge. The overall shape, fit and orientation features between the portable cartridge and the receiving base having a calibrated and known position can then serve as a type of co-registration feature for that particular imaging system.

In this manner a session of imaging on a single living imaging subject can be conducted through a plurality of different imaging systems while the imaging subject remains anesthetized the entire time. Again, such transporting between imaging systems can be done via automated means, such as set forth above, or can be accomplished manually by one or more users, such as by hand carrying the portable cartridge from one imaging system to another and porting the cartridge to suitable accommodating receiving bases at each imaging system. It is specifically contemplated that multiple disparate imaging systems can each have their own separate anesthetic gas providing facilities and orientation features and/or co-registration shapes or marks, such that a single living imaging subject in a single portable cartridge can be anesthetized and imaged in a co-registrable manner in multiple imaging systems. Of course, multiple similar portable imaging subject cartridges can be used within a robust overall multi-modal imaging system.

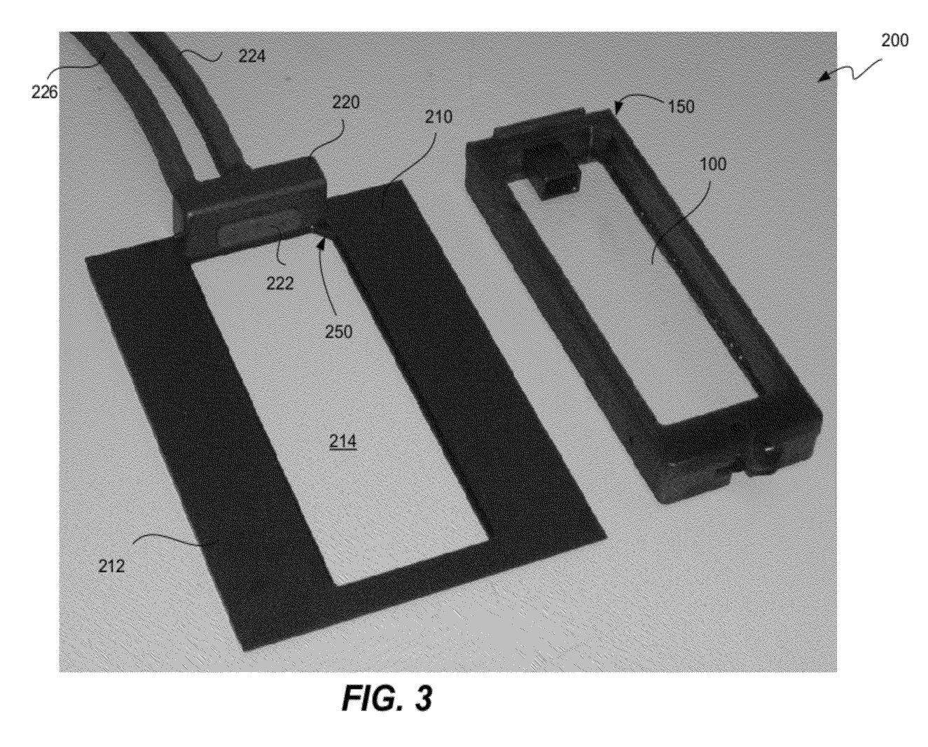

Moving now to FIG. 4 the exemplary portable imaging subject cartridge of FIG. 2 with its lid closed as it is being docked into the receiving base of FIG. 3 is illustrated in obverse perspective view according to one embodiment of the present invention. Imaging subject handling system 201 reflects a portable cartridge 100 having an imaging subject 61 therein and being closed by way of a locked shut locking mechanism 130. From the obverse or bottom view shown, the portable cartridge 100 can be see as being installed within receiving base 210 having an exposed gasket 222 and/or other gas interfacing components adapted to provide an anesthetic gas flow to the portable cartridge as soon as the cartridge is installed.

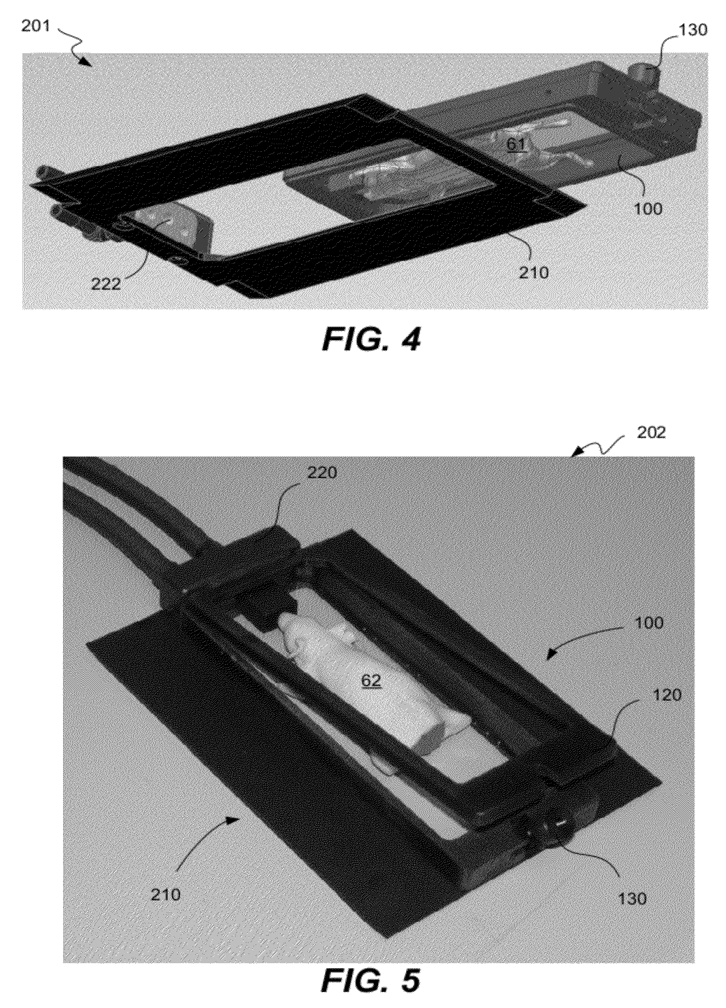

FIG. 5 illustrates in top perspective view an exemplary portable imaging subject cartridge docked into a receiving base and having a dummy imaging subject disposed therein. As shown, imaging subject handling system 202 depicts a portable cartridge 100 having a dummy imaging subject 62 therein and docked into a suitable receiving base 210 and gas processing component 220. Although lid 120 would ordinarily be closed and locking mechanism 130 raised up through the receiving slot and locked onto the lid, the locking mechanism and lid are shown as being open for purposes of illustration here.

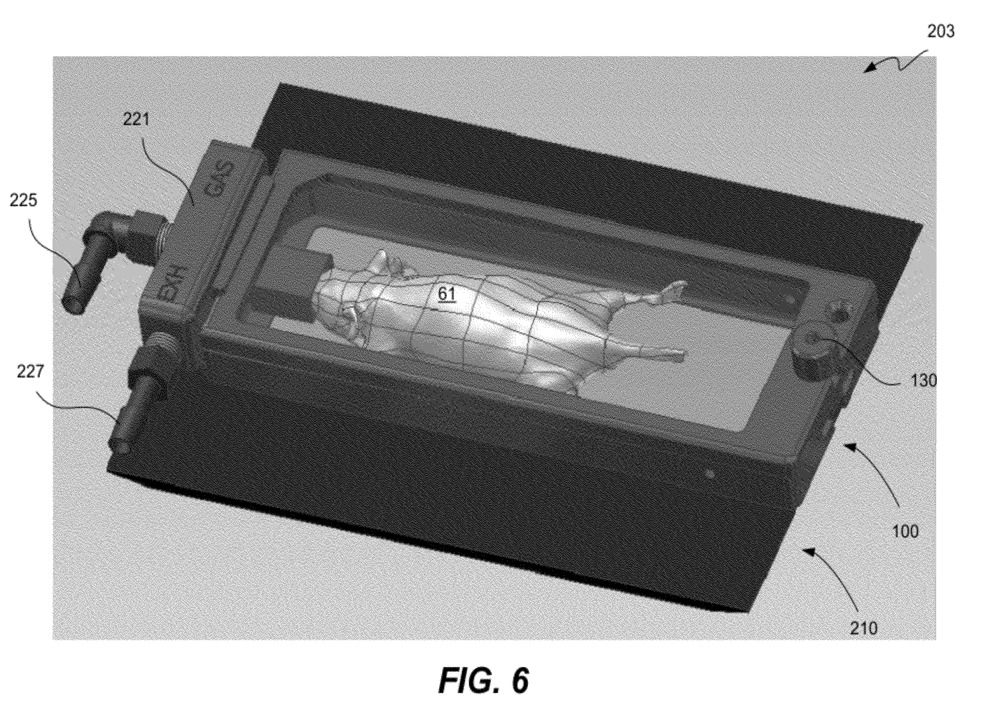

Continuing with FIG. 6, an exemplary portable imaging subject cartridge docked into a receiving base and having an alternative gas porting arrangement is shown in top perspective view. Alternative imaging subject handling system 203 can be substantially similar to the foregoing disclosed systems. In particular, system 203 can include a portable cartridge 100 containing an imaging subject 61 and having a closed locking mechanism 130, as well as a receiving base 210 therefor. A slightly different gas processing component 221 associated with the receiving base 210 can include a gas inlet port 225 and exhaust port 227 that are located at different places than those provided for component 220 described above. Of course, numerous other variations in the arrangements and locations of other components in cartridge 100, receiving base 210 and/or system 200 can also be practiced without departing from the base principles of the invention disclosed herein. In fact, it is specifically contemplated that any and all such variations be included in various ways of practicing the disclosed invention.

### Co-Registration Features

As noted above, one or more orientation features can be used on portable imaging subject cartridges and items or equipment located at the different imaging systems. Such orientation features can facilitate the accurate orientation and placement of a portable cartridge within an imaging system, and could serve in a co-registration capacity in some instances. Although the exemplary mating shapes above involved a triangular corner relief and mating component in the portable cartridge and receiving base, it will be understood that numerous other alternative mating shapes and mechanisms for co-registration can also be used. In fact, different types of imaging systems may be more suitable for use with other types of orientation and/or co-registration features, such that multiple types of features can be used in conjunction with a single portable cartridge or imaging system.

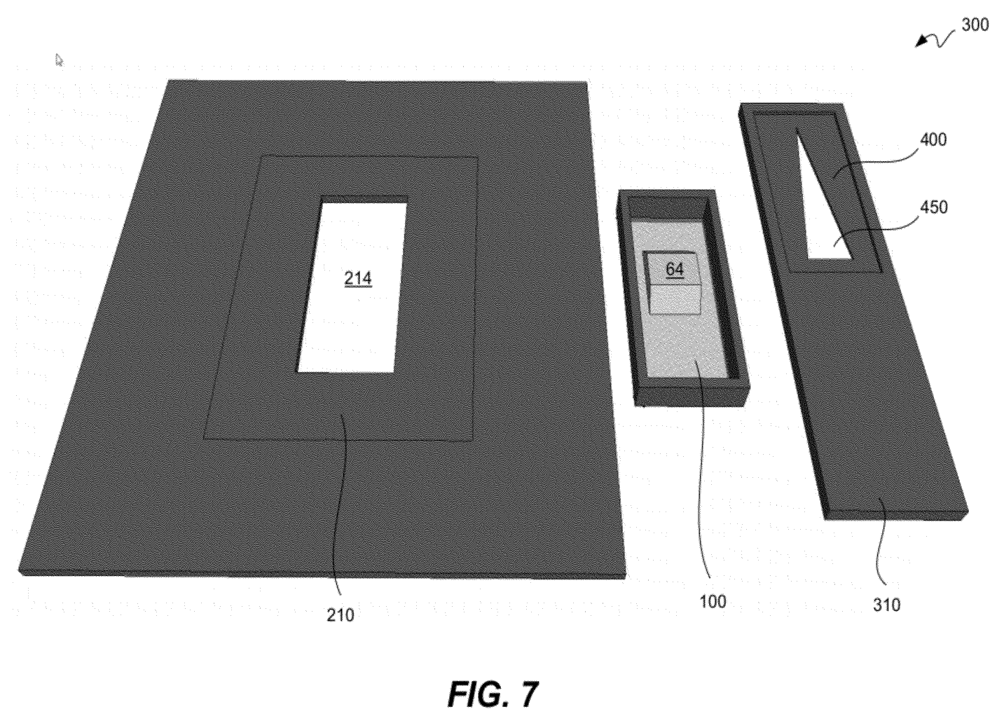

As will be readily appreciated, co-registration features can readily facilitate the merging of images within software from multiple separate imaging systems. Such co-registration features can include shapes, markings and/or other useful items. Further illustration of this concept can be seen in FIG. 7, which shows in top perspective view an exemplary portable cartridge and two different types of receiving bases having different co-registration features therefor according to one embodiment of the present invention. Multi-modal imaging system 300 can include a portable imaging subject cartridge 100 having an imaging subject 64 therein, a first receiving base 210 having an opening 214 adapted to receive the cartridge, and a separate second receiving base 310 that is also adapted to receive the cartridge. Portable cartridge 100 and first receiving base 210 can be identical or substantially similar to the same items set forth in greater detail above, and can have one or more similar physical co-registration features or shapes. First receiving base 210 can be particularly suitable for use in, for example, a light imaging system, such as the light imaging system 40 discussed above.

Alternatively, second receiving base 310 can be more suitable for use in a different type of imaging system, such as a computed tomography system. In such an arrangement, the portable imaging subject cartridge 100 can be placed onto a recessed receiving region 400 of second receiving base 310. Recessed receiving region 400 can similarly have one or more co-registration shapes adapted to mate with a similar shape on the portable cartridge, such as matching triangular corner portions, although these physical co-registration shapes are not shown again here for purposes of simplicity in illustration. More notably, second receiving base 310 can include a specifically sized and shaped opening 450 in the receiving region 400. This opening 450 can be used to selectively shield light, X-rays or other imaging media according to the provided pattern, and thereby help to locate accurately the imaging subject during a particular imaging process, such as a computed tomography process. In effect, opening 450 serves as another type of co-registration feature and is actually a fiducial co-registration pattern when used in such a selectively shielding manner.

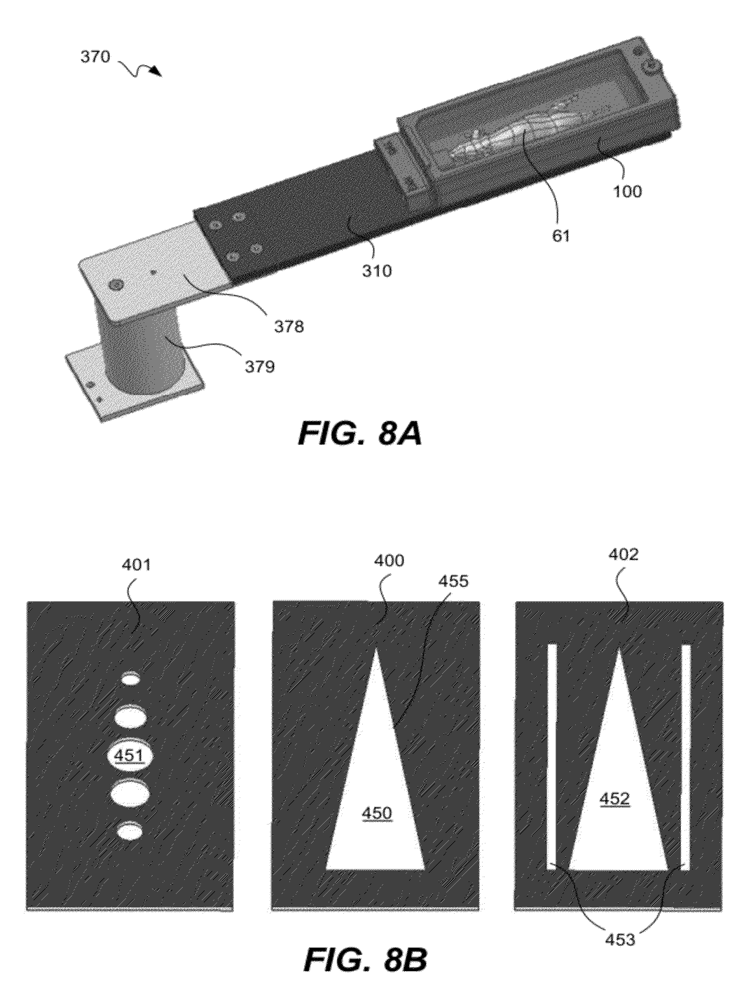

FIG. 8A illustrates in top perspective view an exemplary portion of a portable imaging subject cartridge handling system according to one embodiment of the present invention. Partial handling system 370 can be particularly adapted to handle a portable imaging subject cartridge 100 and imaging subject 61 with respect to an imaging system that utilizes second receiving base 310. As shown, the portable cartridge 100 can be installed onto second receiving base 310, which is then fastened or otherwise coupled to at least a portion of a handling system 370 having an axial component 378 and a rotational component 379. It will be readily appreciated that such a partial handling system 370 can be part of a larger handling system that can involves some portion of automation, manual operation, or any combination thereof, so as to facilitate the accurate installation, placement, movement and/or removal of a portable cartridge into a given imaging system.

Although second receiving base 310 has been initially shown as having a triangular shaped opening 450, it will be understood that numerous other types and shapes of openings can be used in similar ways. FIG. 8B illustrates in top plan view three exemplary fiducial co-registration patterns for a multi-modal imaging system according to various embodiments of the present invention. As noted above, one exemplary receiving region 400 can include a triangular opening 450 having tapered sides or edges 455. An alternative exemplary receiving region 401 can have a plurality of smaller circular openings 451, while still another alternative exemplary receiving region 402 can have a similar triangular opening 452 along with a plurality of narrow side strip openings 453. Each of these various opening patterns can be used effectively as fiducial co-registration patterns during a three-dimensional imaging process, such as a CT process, for example.

With respect to the smaller plurality of circular openings 451, this fiducial co-registration pattern can be quite accurate in physically locating an adjacent imaging subject during a rotational three-dimensional imaging process. One drawback to such a pattern, however, is that the nature of the multiple small shapes can result in a need for a significant amount of constant edge recording and computer processing, which can slow down the overall imaging process. Conversely, the amount of edge recording and computer processing for a single straight-edged pattern, such as triangular opening 450, is substantially smaller and more manageable. This larger opening also gives a wider amount of coverage for the imaging subject, which can be useful in some instances. Similar to the pattern of circular openings 451, the combination pattern of openings 452, 453 results in more complexity in edge recording and processing, and can also involve more complex modeling considerations. Accordingly, triangular opening 450 is thought to be a more practical fiducial co-registration pattern. Nevertheless, given sufficient time and/or computing resources, it is contemplated that any suitable pattern of openings can similarly be used for co-registering a three-dimensional image series under the present invention.

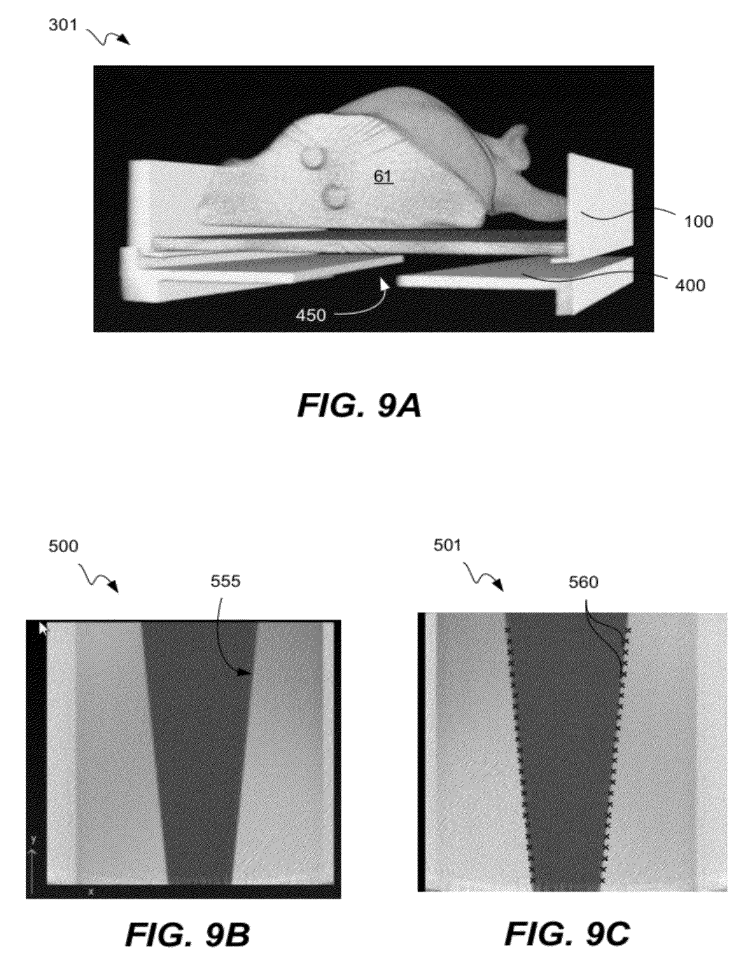

Moving next to FIG. 9A, an imaging subject within an exemplary portable imaging subject cartridge placed atop a triangular fiducial co-registration pattern is illustrated in side cross-sectional view. As will be readily appreciated with respect to the foregoing illustrations and disclosure, multi-modal imaging system 301 can include a portable imaging subject cartridge 100 having an imaging subject 61 contained therein. Cartridge 100 can be located atop a receiving base having a receiving region 400 that includes a triangular opening 450 therein. The opening 450 serves as a fiducial co-registration pattern, and is accordingly preferably located as close to the imaging subject 61 as possible. The shape and nature of the opening can be used to help determine the presumed exact location of the imaging subject for each image in a series of images, particularly where the cartridge and imaging subject are rotated or otherwise moved between images.

FIG. 9B illustrates an exemplary CT image background resulting from the triangular fiducial co-registration pattern of FIG. 9A, while FIG. 9C illustrates an exemplary set of x-points corresponding to the identification of the edge of the feature of FIG. 9B using image processing software. As would be expected, imaging background 500 corresponds to what would be seen in the event that a single two-dimensional x-ray or other similar image is taken with respect to the receiving region 400 having opening 450, as set forth above. Of course, such a simple imaging background reflects an image that is taken when there is no imaging subject present. The darkened area reflects the opening 450 in the receiving region 400, while the lighter areas reflect the shielding material of the receiving region. Straight line 555 corresponds to the tapered edge 455 of the opening.

Computer adjusted imaging background 501 reflects the imaging background 500 having been adjusted according to one or more computer processes. As shown, a plurality of detection points 560 can be overlaid as part of a computer aided imaging analysis or process. As will be readily appreciated, a single image having an imaging subject therein can be substantially more complex than the exemplary images 500, 501 shown here, such that a computerized edge point detection and analysis process should be used. Such a process effectively involves searching for the fiducial layer or pattern as the background in each separate rotated image, such that the remainder of the image having an imaging subject therein can be put into its proper context.

In effect, the search for the fiducial layer or pattern in each image can involve scanning multiple lines along a single axis. For each scanning line, the process can involve detecting the first falling edge and the next rising edge, with the edges being recorded at each iteration. The edges can then be fit into straight lines that most accurately reflect what the known fiducial pattern should be. The goodness or accuracy of the fit and the known rotational angle can then be used to determine whether the proposed straight lines are the actual fiducial pattern or layer. Of course, the number of iterations and confidence level required to make a proper fiducial layer determination can be set as part of the computing analysis parameters, as will be readily appreciated by those skilled in the art. The end result is that the pattern of openings or “fiducial pattern” or layer used can serve as another type of co-registration feature that readily facilitates the overlay of imaging results from different imaging systems.

### Method of Use

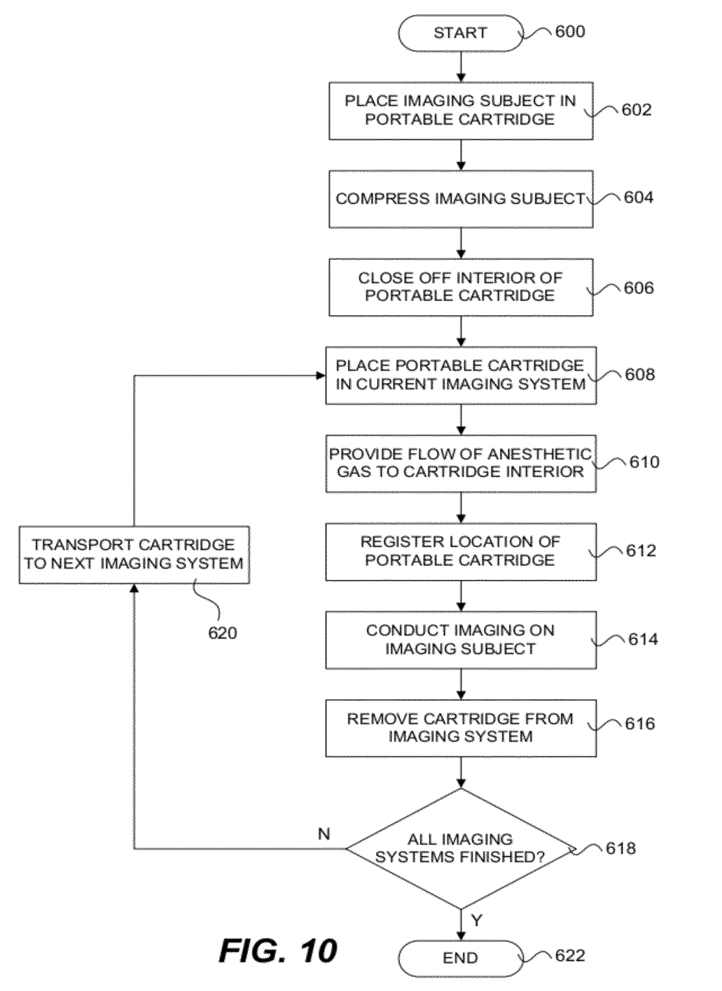

Turning lastly to FIG. 10, a flowchart of an exemplary method of transferring an imaging subject between different imaging systems according to one embodiment of the present invention is provided. While the provided flowchart may be comprehensive in some respects, it will be readily understood that not every step provided is necessary, that other steps can be included, and that the order of steps might be rearranged as desired by a given device manufacturer, vendor or user. For example, step 604 may alternatively be performed after or simultaneously with step 606, or may not even be performed at all in some instances. As another example, the order of steps 610 and 612 could be reversed.

After start step 600, process step 602 can involve the placement of a living imaging subject within a portable imaging subject cartridge, such as that which is set forth in detail above. The living imaging subject can be under the effect of anesthesia prior to the start of the method. At subsequent process step 604, the imaging subject can be compressed, such as between the top and bottom of the portable cartridge. Again, such a compression of the imaging subject is optional. Subsequently, the interior of the portable cartridge can be closed off at process step 606. At the following process step 608, the portable cartridge is placed inside of an imaging system for imaging of the living subject while it remains inside the portable cartridge. After porting with a receiving base inside the imaging system, a flow of anesthetic gas is provided to the portable cartridge interior at subsequent process step 610. In addition, at process step 612 the precise location of the portable cartridge within the imaging system can be registered using one or more co-registration features located on one or more external surfaces of the portable cartridge.

At the following process step 614, imaging can be conducted on the imaging subject while it remains anesthetized within the portable cartridge, after which the portable cartridge can be undocked from the receiving base and removed from the imaging system at process step 616. At subsequent decision step 618 an inquiry can be made as to whether further imaging of that imaging subject is desired at one or more additional imaging systems. If further imaging is desired, then the method continues to process step 620, where the portable cartridge is transported to the next imaging system. The imaging subject can remain compressed and anesthetized within the portable cartridge during this transport step. Method steps 608 through 618 are then repeated for the next imaging system. If no further imaging is desired at decision step 618, however, then the method moves to end step 622, where the method then ends.

Although the foregoing invention has been described in detail by way of illustration and example for purposes of clarity and understanding, it will be recognized that the above described invention may be embodied in numerous other specific variations and embodiments without departing from the spirit or essential characteristics of the invention. Certain changes and modifications may be practiced, and it is understood that the invention is not to be limited by the foregoing details, but rather is to be defined by the scope of the appended claims.

What is claimed is:

1. An imaging subject handling system, comprising:
a first portable imaging subject cartridge adapted to contain and anesthetize a living imaging subject therein during imaging processes within and transport between separate imaging systems, wherein said first portable imaging subject cartridge includes
a gas delivery interface including a gas inlet and gas exhaust, said gas delivery interface being adapted to accept an anesthetic gas flow from an outside source and provide the anesthetic gas to the living imaging subject while the first portable imaging subject cartridge is within one of the separate imaging systems,
one or more walls, a bottom and a separable lid that together define a closed interior and operate to retain substantially all of the anesthetic gas therewithin while the first portable imaging subject cartridge is in transport between separate imaging systems, wherein said at least one of said one or more walls, bottom and lid includes a optically transparent and radiolucent region therethrough, and
a locking mechanism adapted to fasten said separable lid to the remainder of the first portable imaging subject cartridge;
a receiving base adapted to be installed within a chamber of a separate imaging system, wherein said receiving base is configured to receive and interface with said first portable imaging subject cartridge; and
a gas delivery system coupled to said receiving base and configured to deliver the anesthesia gas flow to and receive exhaust from the gas delivery interface of the first portable imaging subject cartridge.

2. The imaging subject handling system of claim 1, wherein said living imaging subject is a mouse.

3. The imaging subject handling system of claim 1, wherein said separable lid is removable from the remainder of the first portable imaging subject cartridge.

4. The imaging subject handling system of claim 1, wherein said first portable imaging subject cartridge is adapted to be carried manually during transport between separate imaging systems.

5. The imaging subject handling system of claim 1, wherein both of the bottom and separable lid of said first portable imaging subject cartridge comprise substantially optically transparent and radiolucent regions, whereby light from a light source located beneath the first portable imaging subject cartridge shines through the first portable imaging subject cartridge and is receivable at an imaging device located above the first portable imaging subject cartridge.

6. The imaging subject handling system of claim 1, wherein said first portable imaging subject cartridge further includes an internal nose cone adapted to receive the nose of the living imaging subject and deliver the anesthesia gas from the gas delivery interface thereto.

7. The imaging subject handling system of claim 1, wherein said one or more walls are dimensioned such that living imaging subject is compressed between the bottom and separable lid when the first portable imaging subject cartridge is closed and the living imaging subject is contained therein.

8. The imaging subject handling system of claim 1, wherein said first portable imaging subject cartridge further includes one or more co-registration features located on the outer surface of at least one of said one or more walls, bottom and lid, and wherein said one or more co-registration features are adapted to facilitate the accurate repeated positioning of the first portable imaging subject cartridge inside multiple separate imaging systems.

9. The imaging subject handling system of claim 1, wherein the imaging subject handling system comprises no ferrous metal parts.

10. The imaging subject handling system of claim 1, wherein said receiving base comprises a gas processing component adapted to receive the anesthetic gas flow from the outside source and deliver the anesthetic gas flow to said gas delivery interface, and also comprises a substantially flat surface having an opening therethrough adapted to receive said first portable imaging subject cartridge therein.

11. The imaging subject handling system of claim 1, further including:
a second portable imaging subject cartridge adapted to contain and anesthetize a living imaging subject therein during imaging processes within and transport between separate imaging systems, wherein said first and second portable imaging subject cartridges are substantially similar except for different distances between the bottom and separable lid of each respective cartridge.

12. A portable imaging subject cartridge adapted to contain and anesthetize a living imaging subject therein during imaging processes within and transport between separate imaging systems, said portable imaging subject cartridge comprising:
a gas delivery interface including a gas inlet and gas exhaust, said gas delivery interface being adapted to accept an anesthetic gas flow from an outside source and provide the anesthetic gas to the living imaging subject while the portable imaging subject cartridge is within one of said separate imaging systems; and
one or more walls, a bottom and a top that together define a closed interior and operate to retain substantially all of the anesthetic gas therewithin while the portable imaging subject cartridge is in transport between separate imaging systems, wherein said at least one of said one or more walls, bottom and top includes an optically transparent and radiolucent region therethrough.

13. The portable imaging subject cartridge of claim 12, further including:
one or more co-registration features located on the outer surface of at least one of said one or more walls, bottom and top, and wherein said one or more co-registration features are adapted to facilitate the accurate repeated positioning of the portable imaging subject cartridge inside multiple separate imaging systems.

14. The portable imaging subject cartridge of claim 12, wherein said top is removable from the remainder of the portable imaging subject cartridge.

15. The portable imaging subject cartridge of claim 12, wherein said portable imaging subject cartridge is adapted to carried manually during transport between separate imaging systems.

16. The portable imaging subject cartridge of claim 12, wherein both of the bottom and top comprise substantially optically transparent and radiolucent regions, whereby light from a light source located beneath the portable imaging subject cartridge shines through the portable imaging subject cartridge and is receivable at an imaging device located above the portable imaging subject cartridge.

17. The portable imaging subject cartridge of claim 12, further including:
an internal nose cone adapted to receive the nose of the living imaging subject and deliver the anesthesia gas from the gas delivery interface thereto.

18. The portable imaging subject cartridge of claim 12, wherein said one or more walls are dimensioned such that living imaging subject is compressed between the bottom and top when the portable imaging subject cartridge is closed and the living imaging subject is contained therein.

19. A method of imaging a living imaging subject in multiple different imaging systems, comprising:
placing the living imaging subject within a portable imaging subject cartridge having a gas delivery interface and also one or more walls, a bottom and a top that together define a closed interior, wherein said at least one of said one or more walls, bottom and top includes an optically transparent and radiolucent region therethrough;
closing off the interior of the portable imaging subject cartridge such that ambient air outside the portable imaging subject cartridge does not substantially mix with gas inside the interior or the portable imaging subject cartridge;
placing the portable imaging subject cartridge within a first imaging system;
providing a flow of anesthetic gas to the interior of the portable imaging subject cartridge while the portable imaging subject cartridge is within the first imaging system;
conducting a first series of imaging on the living imaging subject while the portable imaging subject cartridge is within the first imaging system;
removing the portable imaging subject cartridge from the first imaging system;
transporting the portable imaging subject cartridge from the first imaging system to a second separate imaging system, wherein a portion of the anesthetic gas remains within the interior of the portable imaging subject cartridge during said step of transporting;
placing the portable imaging subject cartridge within the second separate imaging system;
providing a flow of anesthetic gas to the interior of the portable imaging subject cartridge while the portable imaging subject cartridge is within the first imaging system; and
conducting a second series of imaging on the living imaging subject while the portable imaging subject cartridge is within the second imaging system.

20. The method of claim 19, further including the steps of:
registering the exact location of the portable imaging subject cartridge within the first imaging system by reading one or more co-registration features located on the outer surface of at least one of the one or more walls, bottom and top of the portable subject imaging cartridge; and
registering the exact location of the portable imaging subject cartridge within the second imaging system by reading one or more co-registration features located on the outer surface of at least one of the one or more walls, bottom and top of the portable subject imaging cartridge.
